(19) United States
(12) Patent Application Publication
(10) Pub. No.: US 2011/0021777 A1
(43) Pub. Date: Jan. 27, 2011

(54) PROCESS IMPROVEMENT

(75) Inventor: Franz J. WEIBERTH, Bridgewater, NJ (US)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: MILLENNIUM PHARMACEUTICALS, INC., Cambridge, MA (US)

(21) Appl. No.: 12/787,443

(22) Filed: May 26, 2010

Related U.S. Application Data
(63) Continuation of application No. PCT/US2008/084613, filed on Nov. 25, 2008.
(60) Provisional application No. 60/991,280, filed on Nov. 30, 2007.

(51) Int. Cl. C07D 491/044

(52) U.S. Cl. 546/89

(57) ABSTRACT

An improved chemical synthesis for intermediates of compounds having useful biological activity is disclosed, where the use of PBr₃ is employed as a reagent for a selective ring opening of cyclopropylcarbinols to give bromopropylidene products which are highly selectively the E isomer.

### FIELD OF THE INVENTION

This invention is directed to an improvement in synthetic processes for making chemical compounds having useful biological activity.

### BACKGROUND OF THE INVENTION

The present invention is an improvement in the synthetic preparation of 1-{5-[3-bromoprop-(E)-ylidene]-5,11-dihydro-10-oxa-1-azadibenzo[a,d]cyclohepten-7-yl} ethanone, which is an intermediate used for the synthesis of biologically active compounds, for example, those disclosed in U.S. Pat. No. 6,329,385.

### SUMMARY OF THE INVENTION

An improved synthetic preparation of intermediates in the synthesis of compounds having useful biological activity is disclosed, where PBr₃ is employed as a reagent for a selective ring opening of cyclopropylcarbinols to give bromopropylidene products which are highly selectively the E isomer (Scheme 1). The bromopropylidenes can be isolated using standard techniques employed by those skilled in the art. More preferentially, in an application of this technology, the intermediate is not isolated. Instead, it is converted in the same reaction pot to the product named above under Friedel-Crafts conditions (AcCl, AlCl₃, DCM, or other Friedel-Crafts systems), to give, after quenching, extractive work up, and concentration, the product named above in 85% yield as a >18:1 mixture of E/Z isomers. DCM (dichloromethane also known as methylene chloride) is a preferred solvent for these transformations, but other solvents compatible with PBr₃, AcCl and Friedel-Crafts reagents can also be used.

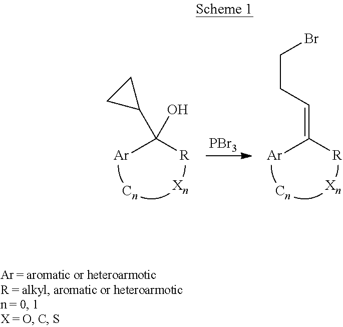

### DETAILED DESCRIPTION OF THE INVENTION

In Scheme 2 is seen the reaction sequence used in the patent cited above.

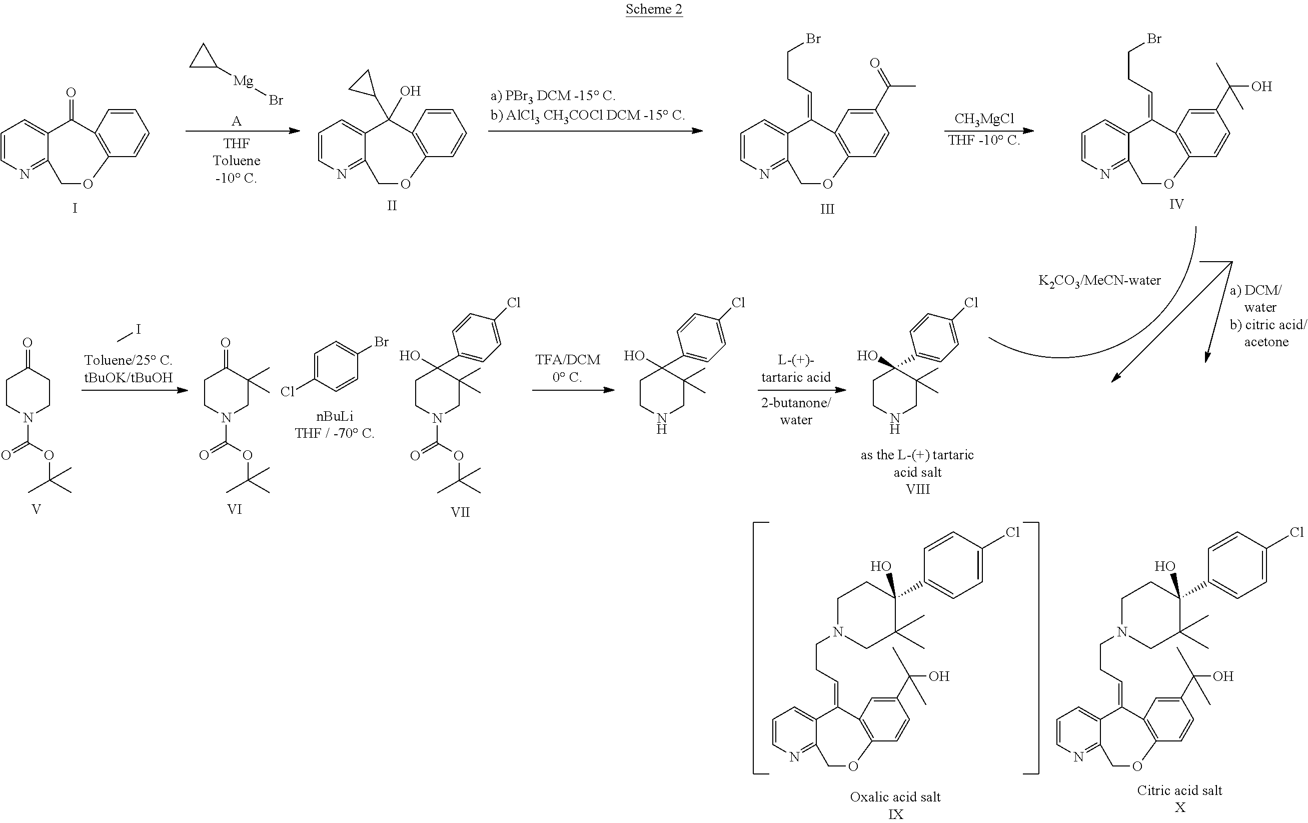

In Scheme 3 is seen the reaction steps improved by the use of PBr₃ as disclosed here.

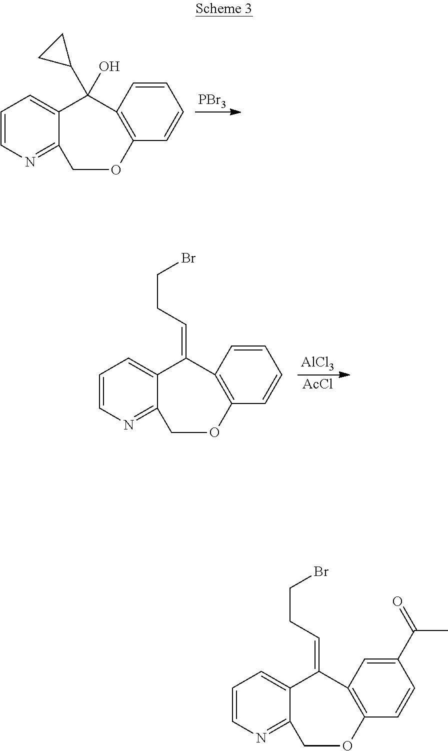

### EXAMPLES

### Example 1

Synthesis of 1-(5-(3-Bromoprop-(E)-ylidene)-5,11-dihyro-10-oxa-1-azadibenzo[a,d]cyclohepten-7-yl)ethanone

### (Scheme 2, Compound III)

5-cyclopropyl-5,11-dihydro-10-oxa-1-azadibenzo[a,d]cyclohepten-5-ol (175 g, 0.691 mole) is suspended in methylene chloride (1750 mL) in a 5 L, 3-necked round-bottom flask equipped with a nitrogen blanket, teflon-coated thermocouple temperature sensor, and a mechanical stirrer, and is cooled to −15° C. Phosphorus tribromide (98.2 g, 0.363 mol, 0.53 eq.) is added using a syringe over a period of 45 minutes while maintaining a temperature of −23 to −15° C. The reaction mixture is stirred for 30 minutes at −30 to −15° C. until all the solids are dissolved. The solution is allowed to warm to 5 to 10° C. over a period of 1 hour. Additional phosphorus tribromide (5.5 g, 0.03 eq.) is added. The yellow suspension is cooled to −5° C. and AlCl₃ (230.3 g, 1.73 mol, 2.5 eq.) is added over a period of 2 to 3 minutes. Acetyl chloride (54.23 g, 0.69 mol, 1.00 eq.) is added while maintaining a temperature of 1 to 6° C. The mixture is quenched into a mixture of ice (1.6 kg) and water (2.6 L) and the phases are separated. The aqueous phase is extracted with methylene chloride (0.5 L). The combined organic phase is filtered through celite, is washed with NaHCO₃ (0.65 L of 5% aqueous solution), is dried and filtered over Na₂SO₄ (0.25 kg), and is concentrated to afford 1-[5-(3-bromo-propylidene)-5,11-dihydro-10-oxa-1-azadibenzo[a,d]cyclohepten-7-yl]-ethanone (227.7 g) as a thick, light-orange oil. 210.2 g (84.9% overall yield for the 2-step concatenated sequence) is corrected for methylene chloride (7.7% by weight) (NMR), ca. 17:1 ratio of acylated E/Z isomers by HPLC.

HPLC Conditions: Column: 4.6×150 mm Eclipse XDB-C8, 5 μ; Flow: 1.0 mL/minute; Mobile Phase: acetonitrile/0.1% TFA (40:60), Column Temperature=35° C.; UV detection at 250 nm; 5-cyclopropyl-5,11-dihydro-10-oxa-1-azadibenzo[a,d]cyclohepten-5-ol=2.2 minutes; Z-acylated intermediate 1-[5-(3-bromo-propylidene)-5,11-dihydro-10-oxa-1-azadibenzo[a,d]cyclohepten-7-yl]-ethanone =5.9 min; Z-unacylated intermediate 5-(3-bromo-propylidene)-5,11-dihydro-10-oxa-1-azadibenzo[a,d]cycloheptene =6.2 min; E-acylated 1-[5-(3-bromo-propylidene)-5,11-dihydro-10-oxa-1-azadibenzo[a,d]cyclohepten-7-yl]-ethanone=7.2 min; E-unacylated intermediate 5-(3-bromo-propylidene)-5,11-dihydro-10-oxa-1-azadibenzo[a,d]cycloheptene=9.0 min.

¹H NMR: (CDCl₃) δ 8.54 (1 H, d), 7.95 (1 H, s), 7.75 (1 H, d), 7.57 (1 H, d), 7.32 (1 H, m), 6.86 (1 H, d), 6.15 (1 H, t), 5.7-5.3 (2 H, br m), 3.47 (2 H, t), 2.75 (2 H, m), 2.55 (3 H, s) ppm. ¹³C NMR: (CDCl₃) 196.57, 159.18, 152.72, 148.79, 138.63, 135.65, 135.36, 131.24, 130.86, 130.69, 130.03, 126.06, 123.74, 119.72, 72.14, 32.13, 32.04, 26.34 ppm.

### Example 1a

Synthesis of 1-(5-(3-Bromoprop-(E)-ylidene)-5,11-dihyro-10-oxa-1-azadibenzo[a,d]cyclohepten-7-yl)ethanone

### (Scheme 2, Compound III)

5-cyclopropyl-5,11-dihydro-10-oxa-1-azadibenzo[a,d]cyclohepten-5-ol (225 g, 0.888 mole, 1 eq.) is suspended in methylene chloride (2250 mL) in an 8 L, jacketed dry glass reactor equipped with a mechanical stirrer and purged with nitrogen, and is cooled to -15° C. Phosphorus tribromide (126 g, 44 mL, 0.46 mole, 0.523 eq.) is added dropwise at −15° C. using a dropping funnel (100 m) over a period of 1.25 hours. The dropping funnel is flushed with dichloromethane (140 mL) and the reactor is warmed to −5° C. and is stirred for 1.5 hours. Aluminum trichloride (295 g, 2.212 mol, 2.5 eq.) is added progressively over the period of 1 hour, wherein an exotherm is observed. The reaction mixture is stirred for an additional 15 minutes. Acetyl chloride (66 g, 66 mL, 0.84 mol, 0.946 eq.) is added dropwise over 1 hour at —5° C. via dropping funnel (100 mL). The reaction mixture is stirred for an additional 1.5 hours. Water (4500 mL) is charged very slowly for the first 200 mLs. The remaining water is charged faster. The temperature is raised to 20° C. by the end of the hydrolysis. The content of the reactor is filtered through a bed of celite (170 g) and the filter cake is washed with methylene chloride (250 mL). The combined filtrate is settled and after separation of the layers, the lower organic layer is washed with sodium hydrogen carbonate (2200 mL, saturated aqueous solution) and then with sodium chloride (2200 mL, 10% aqueous solution). The solvent (2250 mL) is distilled under atmospheric pressure. t-butyl methyl ether (1400 mL) is added over 10 minutes. The distillation is continued until 700 mL of the concentrate remains (about 1200 mL is distilled). The reactor is cooled slowly to 20° C. and is maintained at 20° C. while stirring for about 2 hours. The resulting slurry is filtered and the filter cake is washed with t-butyl methyl ether (225 mL). The damp filter cake is dried in a vacuum oven (40° C.) to afford 1-[5-(3-bromopropylidene)-5,11-dihydro-10-oxa-1-azadibenzo[a,d]cyclohepten-7-yl]-ethanone (261.7 g, 82.2% Yield). HPLC Area =98.4%.

The present invention may be embodied in other specific forms without departing from the spirit or essential attributes thereof.

What is claimed is:

1. An improved ring-opening of cyclopropylcarbinols to give bromopropylidene products which are highly selectively the E isomer, comprising: treating a cyclopropylcarbinol with PBr₃.

2. An improved synthesis of 5-(3-bromoprop-(E)-ylidene]-5,11-dihydro-10-oxa-1-azadibenzo [a,d] cycloheptene, comprising treating 5-cyclopropyl-5,11-dihydro-10-oxa-1-azadibenzo[a,d]cyclohepten-5-ol with PBr₃ in a compatible solvent.

3. The synthesis according to claim 2, wherein the solvent is dichloromethane.

4. An improved synthesis of 1-{5-[3-bromoprop-(E)-ylidene]-5,11-dihydro-10-oxa-1-azadibenzo[a,d]cyclohepten-7-yl} ethanone, comprising:
carrying out the reaction:

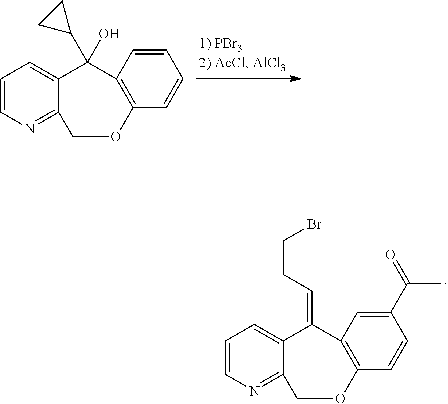

5. The synthesis according to claim 4, wherein the reaction is carried out with dichloromethane as solvent.
